(19) United States
(12) Patent Application Publication
(10) Pub. No.: US 2011/0229920 A1
(43) Pub. Date: Sep. 22, 2011

(54) SCREENING METHODS FOR IDENTIFYING SPECIFIC STAPHYLOCOCCUS AUREUS INHIBITORS

(75) Inventors: Richard H. Guenther, Cary, NC (US); Samuel P. Yenne, Raleigh, NC (US)

(73) Assignee: TRANA DISCOVERY, INC., Cary, NC (US)

(21) Appl. No.: 13/121,381

(22) PCT Filed: Sep. 24, 2009
(86) PCT No.: PCT/US09/58208

§ 371 Date: Jun. 7, 2011

Related U.S. Application Data
(60) Provisional application No. 61/100,996, filed on Sep. 29, 2008.

(51) Int. Cl. C12Q 1/42; C07H 21/02; C12N 9/00; C12N 9/08; C12N 9/16; C07H 21/00; G01N 33/48; C12Q 1/28

(52) U.S. Cl. 435/21; 536/23.1; 435/183; 435/192; 435/196; 536/25.4; 436/94; 435/28

(57) ABSTRACT

Methods of inhibiting S. aureus propagation, and screening for compounds that inhibit S. aureus propagation, are described. A method of inhibiting S. aureus propagation comprises inhibiting ribosomal binding of a specific S. aureus tRNA in the S. aureus by an amount sufficient to inhibit S. aureus protein expression. A method of screening for compounds useful for inhibiting S. aureus propagation comprises contacting a specific S. aureus tRNA to a ribosome that binds that tRNA in the presence of the test compound, and then determining whether the compound inhibits the binding of that tRNA.

### CROSS-REFERENCE TO RELATED APPLICATION

This application claims the benefit under 35 U.S.C. §119 of U.S. Provisional Patent Application No. 61/100,996 filed Sep. 29, 2008. The disclosure of said U.S. Provisional Patent Application No. 61/100,996 is hereby incorporated herein by reference, in its respective entirety, for all purposes.

### FIELD OF THE INVENTION

The present invention concerns antibacterial agents that are directed against tRNA targets, particularly, tRNA targets specific for Staphylococcus aureus, and methods of screening for antibacterial and agents directed against such tRNA targets.

### BACKGROUND OF THE INVENTION

The need to discover new classes of antibiotic compounds and/or antibiotics with different target sites is being reiterated frequently with the threat of drug resistant pathogens, reemerging pathogens and/or bio-terrorism concerns. With each passing decade, strains of virtually all important bacterial pathogens of humans have arisen that are resistant to at least one class of antibiotics, and strains resistant to multiple classes of antibiotics have become increasingly widespread. In fact, according to the Centers for Disease Control and Prevention (CDC 2000-2001), virtually all significant bacterial infections in the world are becoming resistant to the antibiotic treatment of choice. This rise is generally attributed to pathogens which have become resistant to commonly used antibiotics which focus on a limited number of target sites. Some pathogens that were generally considered historical disease causing agents are reemerging either due to genetic modifications making the organism more virulent and/or exposure to a larger portion of the world population. Related to the naturally occurring genetic modifications are intentional genetic modifications conducted by groups with bio-terrorist desires. Frequently, these intentional genetic modifications will focus on making an otherwise susceptible disease pathogen resistant to the current antibiotics with known target sites.

For Staphylococcus aureus in particular, evolving resistance mechanisms have created significant treatment challenges over the years. Beginning with penicillinase-producing strains that were resistant to conventional penicillins, the need for newer, effective antibiotics against this organism has ensued. Most recently, with the emergence of community-associated methicillin-resistant S. aureus (CA-MRSA), traditional first-line antibiotics are once again ineffective, and, unfortunately, the prevalence of CA-MRSA is already high, accounting for well over 60% of all cases of community-associated S. aureus infections. Even worse is the recent discovery of multi-drug resistant strains of CA-MRSA that are capable of rapidly acquiring resistance to most all available agents via a plasmid-mediated mechanism. In the face of this threat, a number of new antibiotics targeted against S. aureus are under development. However, of the new antibiotics known to be in clinical development, none has a novel mechanism of action. For these reasons the need for totally new treatments for S. aureus infections is evident.

It would be advantageous to have a method for identifying compounds useful for treating S. aureus infections via a new mechanism. The present provides such a method, as well as treatment methods using the discovered compounds.

### SUMMARY OF THE INVENTION

Methods for identifying specific inhibitors of S. aureus, isolated tRNA fragments that are useful in these methods, and kits including these fragments, are disclosed. Also disclosed are methods of treating and/or preventing S. aureus infection using the inhibitors of S. aureus propagation, and pharmaceutical compositions including the inhibitors and a pharmaceutically-acceptable carrier. Combination therapy using one or more of the inhibitors, and a second anti-bacterial compound, are also disclosed.

The inhibition of S. aureus propagation results from inhibition of S. aureus protein synthesis. Specifically inhibition refers to the selective inhibition of S. aureus in the presence of other bacteria. One advantage of selective inhibition of S. aureus is that, in this manner, one can treat bacterial infections without the concomitant development of antibacterial resistance in the colon, and the side effects resulting from disturbing beneficial bacteria in the colon, such as diarrhea, CDAD, pseudomembranous colitis, and the like.

Methods for screening inhibitors of S. aureus propagation may involve forming a mixture comprising a linear sequence of a tRNA anticodon stem loop fragment, a ribosome capable of binding to the tRNA anticodon stem loop fragment, and a test compound. The mixture is incubated under conditions that allow binding of the tRNA anticodon stem loop fragment and the nucleic acid molecule in the absence of the test compound. One can then determine whether or not a test compound inhibits the propagation of S. aureus. Inhibition of binding of the tRNA ASL fragment and the ribosome is indicative of the test compound being an inhibitor of S. aureus propagation.

S. aureus uses six different tRNA to incorporate arginine into a growing protein. Other bacteria use five different tRNA to incorporate arginine into the growing protein. The methods described herein use the tRNA specific for arginine incorporation in S. aureus in a screening method, where the tRNA forms a complex. Specific inhibitors of S. aureus will interrupt the complex, and therefore prevent arginine incorporation into the growing protein/peptide, which results in bactericidal action. Once inhibitors are identified, they can be tested in vitro for specific S. aureus activity either by incubating them with other tRNA/ribosome complexes, to see if they do or do not disrupt these complexes. Alternatively, one can incubate the inhibitors with a variety of bacteria and identifying those specific inhibitors of arginine incorporation which are not bactericidal to other bacteria, for example, beneficial bacteria.

Kits for screening inhibitors of the various processes described above are also disclosed. The kits comprise a nucleic acid molecule consisting essentially of a linear sequence of a tRNA anticodon stem loop fragment; and a detectable label.

Compounds which are inhibitors of the various processes described above can be used in methods of treating and/or preventing an S. aureus infection. Such methods are also within the scope of the invention. Pharmaceutical compositions useful in these methods are also within the scope of the invention. Such pharmaceutical compositions include one or more inhibitors, as described herein, and a pharmaceutically-acceptable carrier. Combination therapy, using additional antibacterial compounds which function by a different mechanism, are also disclosed.

The foregoing and other objects and aspects of the present invention are explained in detail in the drawings herein and the specification set forth below.

### BRIEF DESCRIPTION OF THE DRAWINGS

FIG. 1 is a graphic illustration showing the synthesis of proteins via the processes of transcription and translation.

FIG. 2 is a graphic illustration showing “spacefill” and “wire” displays of the structure of tRNA^{Lys} ASL (red) bound to the ribosome (green) at the translocation site. The blue nucleotide embedded in the binding site is the modified nucleotide t⁶A₃₇ on the ASL.

FIG. 3 is a graphic illustration of examples of modified nucleotide bases along bottom row with modifications in circles. Unmodified RNA bases uracil and adenine (in box) are included for comparison. Along the top row are amino acids with functional R-group equivalence to the corresponding nucleotide base in the bottom row.

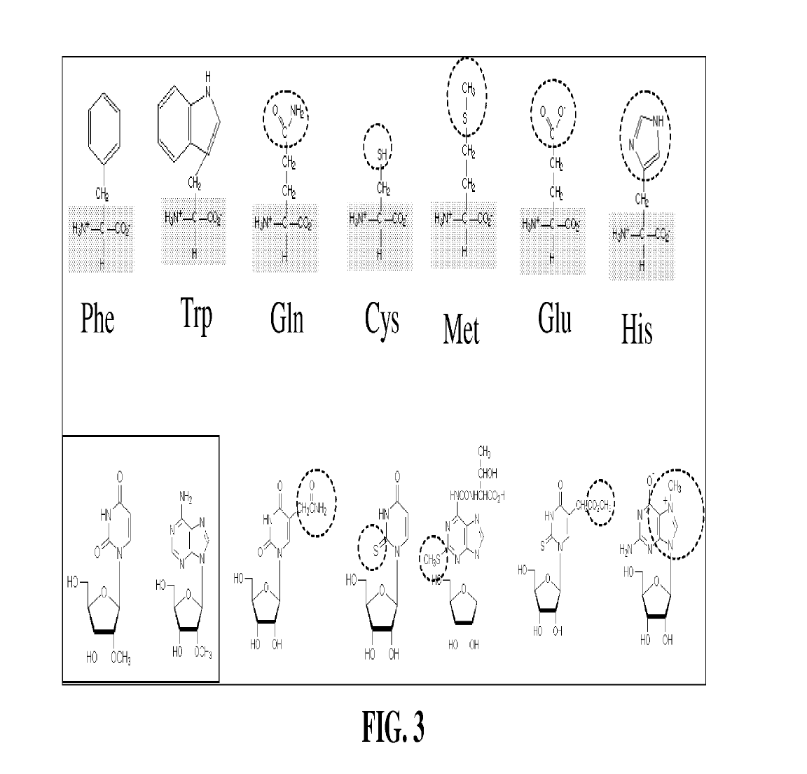

FIG. 4 is an illustration of a synthetic oligomer representing the anticodon stem loop from tRNA^{Arg} in S. aureus. Only when the modifications are present will the ASL bind to programmed ribosomes isolated from S. aureus, which binding can be detected by monitoring the change in fluorescence.

FIG. 5 is a chemical drawing showing the protection of the modified nucleotide bases prior to synthesis of the RNA oligomer. Panel A illustrates protection with trifluoryl acetic acid. Panel B illustrates protection with benzoyl. Panel C outlines the major steps in the conversion of adenosine to t6A. And, panel D illustrates the general protection scheme of the ribose hydroxyl groups.

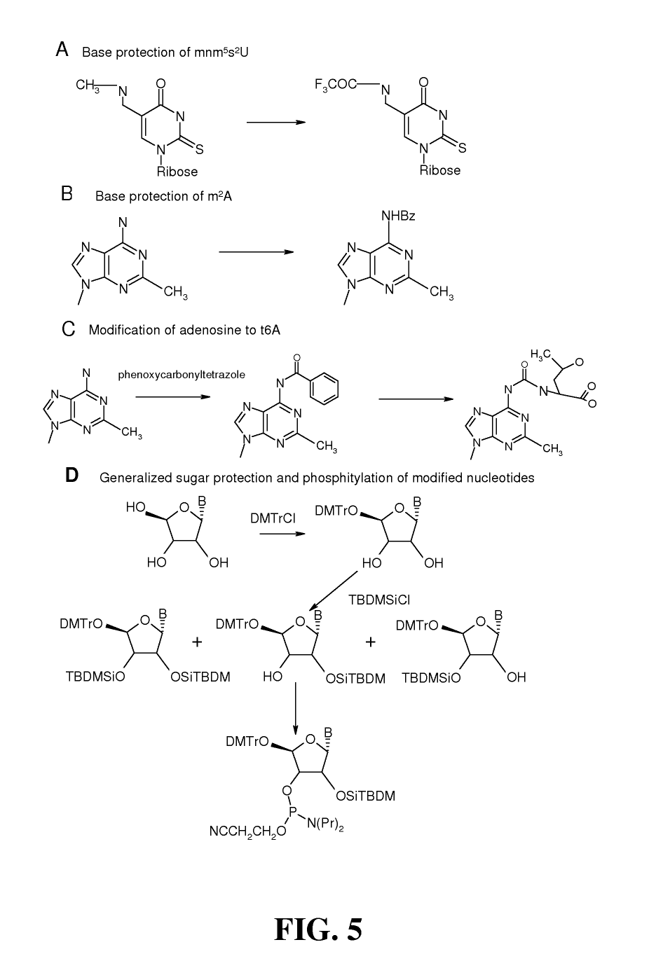

FIG. 6 is a chemical illustration of a Bz-A-CE RNA phosphoramidite monomer showing the Bz (“benzyl”) protecting group on the base and the Silyl sugar protecting group TBDMS (“t-butyldimethylsilane”).

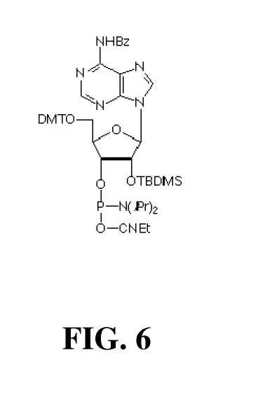

FIG. 7 is a chemical illustration showing the complete synthesis cycle for tRNA synthesis, including de-blocking (A), base condensation (B), capping (C), and oxidation (D) are illustrated. This cycle is completed once for each additional base desired.

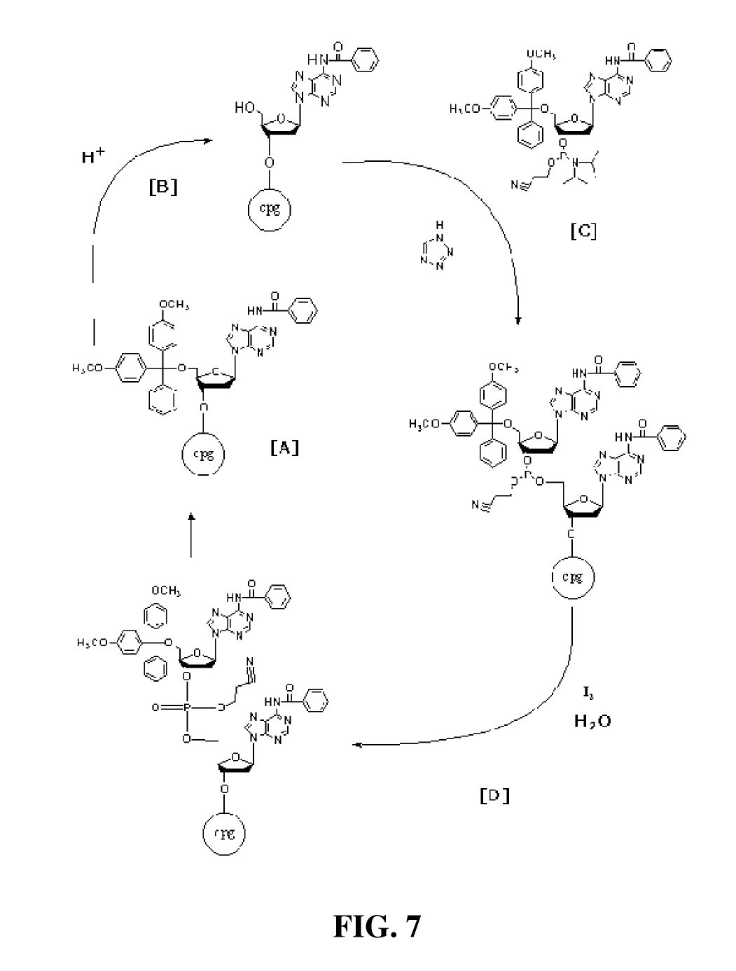

FIG. 8 is a graphic illustration showing the cleavage of a group protecting a heterocyclic primary amine and a cyanoethyl group, using concentrated ammonia, as typically performed in DNA oligonucleotide synthesis.

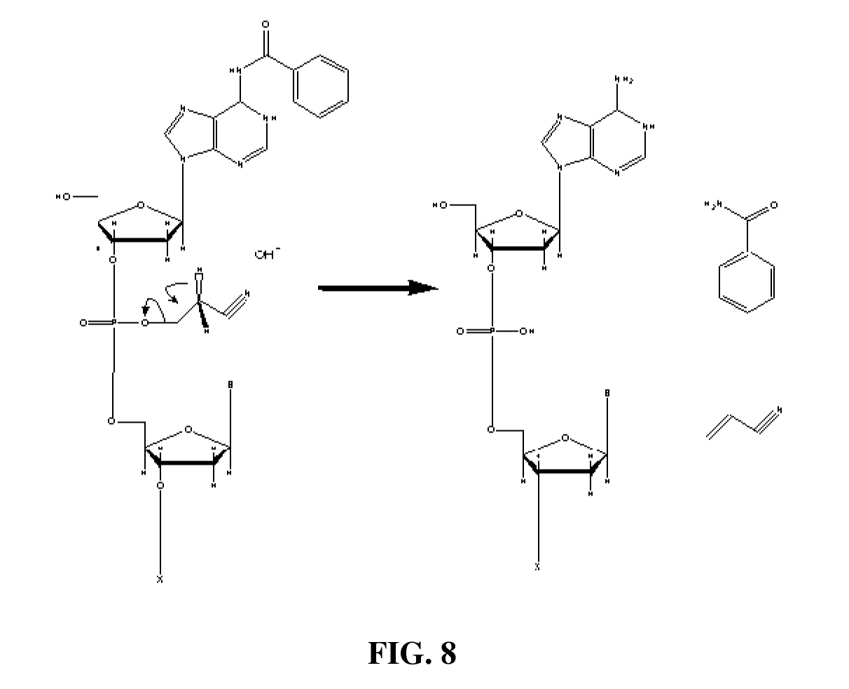

FIG. 9 is a chart showing the titration of fluorescently labeled ASL with programmed ribosomes (dashed line) and unprogrammed ribosomes (solid line).

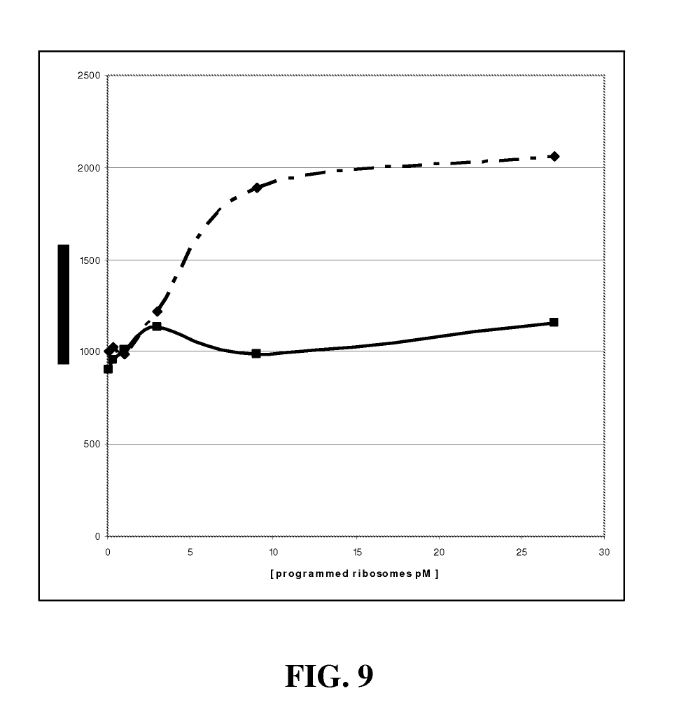

FIG. 10 is a chart showing the chemical diversity of a representative 100 k compound library.

### DETAILED DESCRIPTION

The present invention relates to compositions and methods for identifying compounds useful for specifically inhibiting S. aureus propagation, as well as pharmaceutical compositions and methods for treating S. aureus infections by inhibiting S. aureus propagation. S. aureus propagation can be inhibited by inhibiting translation of S. aureus RNA into proteins.

Prior to describing this invention in further detail, however, the following terms will first be defined.

Definitions

As used herein, an “inhibitor” refers to any compound capable of preventing, reducing, or restricting S. aureus propagation. An inhibitor may inhibit S. aureus propagation, for example, by preventing, reducing or restricting S. aureus protein formation, specifically by inhibiting arginine incorporation into a growing protein strand, and, more specifically, by inhibiting arginine incorporation by focusing on disrupting a complex formed by a tRNA specific for S. aureus' incorporation of arginine into a growing protein strand. In some embodiments, the inhibition is at least 20% (e.g., at least 50%, 70%, 80%, 90%, 95%, 98%, 99%, 99.5%) of the S. aureus propagation as compared to the propagation in the absence of the inhibitor. In one aspect, an inhibitor prevents, reduces, or restricts the binding of a tRNA, or fragment thereof, to a ribosome, preferably a ribosome associated with protein and peptide synthesis. More particularly, the binding is related to the incorporation or arginine into a growing peptide or protein, and, most particularly, the binding is specific for the incorporation of arginine into a protein or peptide encoded by S. aureus, and the tRNA is not useful for the incorporation of arginine into proteins or peptides of other bacteria.

The selection of ASL Arg over ASLs Lys or Glu or Gln reflects an improvement in target selection. A recent analysis of bacterial codon usage including S. aureus (Rocha 2004) has found a high degree of bias. While 6 codons exist for arginine, only two of them are used in high frequency. By designing an assay that selectively looks to inhibit the target tRNA, an added selectivity of the therapeutic can be expected. In addition, a concern in targeting of tRNA Lys function raises the potential for inhibition of human mitochondrial function. The selection of a UCU anticodon as the therapeutic target has an advantage in that it eliminates that concern in that in human mitochondria, the AGA codon is not an amino acid encoding triplet, but rather a stop codon (Kirino 2005).

As used herein, a “label” or “detectable label” is any composition that is detectable, either directly or indirectly, for example, by spectroscopic, photochemical, biochemical, immunochemical, or chemical means. Useful labels include, but are not limited to, radioactive isotopes (for example, 32p, 35S, and 3H), dyes, fluorescent dyes (for example, Cy5 and Cy3), fluorophores (for example, fluorescein), electron-dense reagents, enzymes and their substrates (for example, as commonly used in enzyme-linked immunoassays, such as, alkaline phosphatase and horse radish peroxidase), biotin-streptavidin, digoxigenin, or hapten; and proteins for which antisera or monoclonal antibodies are available. Moreover, a label or detectable moiety can include an “affinity tag” that, when coupled with the target nucleic acid and incubated with a test compound or compound library, allows for the affinity capture of the target nucleic acid along with molecules bound to the target nucleic acid. One skilled in the art will appreciate that an affinity tag bound to the target nucleic acid has, by definition, a complimentary ligand coupled to a solid support that allows for its capture. For example, useful affinity tags and complimentary partners include, but are not limited to, biotin-streptavidin, complimentary nucleic acid fragments (for example, oligo dT-oligo dA, oligo T-oligo A, oligo dG-oligo dC, oligo G-oligo C), aptamers, or haptens and proteins for which antisera or monoclonal antibodies are available. The label or detectable moiety is typically bound, either covalently, through a linker or chemical bound, or through ionic, van der Waals or hydrogen bonds to the molecule to be detected.

The term “host” as used herein refers to human or animal cells or tissues in vitro and human or animal subjects (e.g., avian or mammalian cells, tissues and subjects such as chickens, turkeys, mouse, rat, cats, dogs, cows, pigs, horses, etc.).

The term “ribosome” as used herein refers to both intact active ribosomes and ribosome subunits that retain tRNA binding, such as 30S subunits.

The specific tRNA referred to herein with respect to S. aureus tRNA is preferably a unique or unusual tRNA: that is, one that contains one or more modified bases other than adenine, guanine, cytosine, or uracil in the anticodon binding region (including both the stem and loop thereof), and/or preferably a tRNA that is only found in S. aureus for binding to a corresponding amino acid (e.g., arginine) during protein translation in S. aureus. That is, preferably, the tRNA is specific for arginine, and is only found in S. aureus, or at least is not found in the vast majority (i.e., greater than 90%, preferably greater than 95%, and, ideally, greater than 99% of bacteria other than S. aureus).

Preferably the modified base or bases is/are a nucleotide(s) that is/are at a binding site as described below (e.g., nucleotides 27 through 43) and participates in the binding event. Where carried out in vivo, the tRNA for the corresponding amino acid bound by the S. aureus tRNA preferably does not have the same modified base at the binding site or corresponding nucleotide in the host organism (i.e., pathogen specific modification). Many of these exist in the human host and in agronomically important animal hosts as set forth above). Examples of modified bases are set forth below.

I. tRNA Fragments Useful in the Methods Described Herein

The tRNA fragments (or “tool tRNA fragments”) for use in the screening methods described herein are tRNA fragments from S. aureus that code for arginine, and which have the formula below:

AUGGCs2CUmnm5UCUt6AAGCCAU-label

In one aspect, the tRNA fragment comprises the nucleic acid sequence AUGGCs2CUmnm5UCUt6AAGCCAU-Fluorescein.

In another aspect, the tRNA fragment above can be modified with one or more modified nucleosides, so long as it maintains its selectivity for arginine, and the modified tRNA is still specific for S. aureus over other bacteria. In one aspect, the tRNA fragment incorporates one, two, three, or more modified nucleosides into the nucleic acid sequence. In another aspect, the tRNA fragments incorporate three modified nucleosides into their nucleic acid sequence. Modified nucleosides that can be incorporated into the tRNA fragments include any modified nucleotide, including, but not limited to unknown modified adenosine (?A), 1-methyladenosine (m1A), 2-methyladenosine (m2A), N⁶-isopentenyladenosine (i6A), 2-methylthio-N⁶-isopentenyladenosine (ms2i6A), N⁶-methyladenosine (m6A), N⁶-threonylcarbamoyladenosine (t6A), N⁶-methyl-N⁶ threonylcarbomoyladenosine (m6t6A), 2-methylthio-N⁶-threonylcarbamoyladenosine (ms2t6A), 2′-O-methyladenosine I Inosine (Am), 1-methylinosine Ar(p) 2′-O-(5-phospho)ribosyladenosine (m1I), N⁶-(cis-hydroxyisopentenyl)adenosine (io6A), Unknown modified cytidine (?C), 2-thiocytidine (s2C), 2′-O-methylcytidine (Cm), N⁴-acetylcytidine (ac4C), 5-methylcytidine (m5C), 3-methylcytidine (m3C), lysidine (k2C), 5-formylcytidin (f5C), 2′-O-methyl-5-formylcytidin (f5Cm), unknown modified guanosine (?G), 2′-O-(5phospho) ribosylguanosine (Gr(p)), 1-methylguanosine (m1G), N²-methylguanosine (m2G), 2′-O-methylguanosine (Gm), N²N²-dimethylguanosine (m22G), N²,N²,2′-O-trimethylguanosine (m22Gm), 7-methylguanosine (m7G), archaeosine (fa7d7G), queuosine (Q), mannosyl-queuosine (manQ), galactosylqueuosine (galQ), wybutosine (yW), peroxywybutosine (02yW), unknown modified uridine (?U), 5-methylaminomethyluridine (mnm5U), 2-thiouridine (s2U), 2′-O-methyluridine (Um), 4-thiouridine (s4U), 5carbamoylmethyluridine (ncm5U), 5-methoxycarbonylmethyluridine (mcm5U), 5methylaminomethyl-2-thiouridine (mnm5s2U), 5-methoxycarbonylmethyl-2-thiouridine (mcm5s2U), uridine 5-oxyacetic acid (cmo5U), 5-methoxyuridine (mo5U), 5carboxymethylaminomethyluridine (cmnm5U), 5-carboxymethylaminomethyl-2-thiouridine (cmnm5s2U), 3-(3-amino-3-carboxypropyl)uridine (acp3U), 5-(carboxyhydroxymethyl)uridinemethyl ester (mchm5U), 5-carboxymethylaminomethyl-2′-O-methyluridine (cmnm5Um), 5-carbamoylmethyl-2′-O-methyluridine (ncm5Um), Dihydrouridine (D), pseudouridine (ψ), 1-methylpseudouridine (m1ψ), 2′-O-methylpseudouridine (ψm), ribosylthymine (m5U), 5-methyl-2-thiouridine (m5s2U), and 5,2′-O-dimethyluridine (m5Um).

The tRNA fragment may also be any length of a fragment from a tRNA. In one aspect, the tRNA fragment comprises a fragment of between 9 to 15 continuous nucleotides of a tRNA, 10 to 14 continuous nucleotides of a tRNA, or between 11 to 13 continuous nucleotides of a tRNA. In another aspect, the fragment is a fragment of 8, 9, 10, 11, 12, 13, 14, 15, or 16 continuous nucleotides of a tRNA. In a further aspect, the fragment is a fragment of 12 continuous nucleotides of a tRNA.

The tRNA fragment may or may not be capable of forming a secondary structure. In a one aspect, the tRNA fragment is not capable of forming a stem loop structure with itself. In another aspect, the fragment is a linear fragment of a tRNA that is not capable of forming a stem loop structure with itself.

The tRNA fragment may also be linked to additional nucleic acids. For example, the tRNA fragment may be linked to one or more additional nucleic acids depending on the assay method. In one aspect, the tRNA fragment may be linked to nucleotides used to attach the fragment to a solid support surface. In another aspect, the fragment tRNA is linked to additional nucleic acid molecules at one or both terminal end of the tRNA fragment. In another aspect, the fragment tRNA is linked to additional nucleic acid molecules at both terminal ends. The additional nucleic acid sequences can be any length, preferably between 8 and 16 nucleotides, between 10 and 14 nucleotides, more preferably 12 nucleotides in length. In one aspect, the terminal sequences do not allow the tRNA fragment to form a secondary structure, such as a hairpin loop structure.

The specific tRNA referred to herein with respect to host tRNA is also preferably a unique or unusual tRNA: that is, one that contains one or more modified bases other than adenine, guanine, cytosine, or uracil in the anticodon binding region (including both the stem and loop thereof), as set forth above, and/or preferably one that is the only tRNA available in that host for binding to RNA for priming of translation of S. aureus proteins.

The region of the tRNA to which binding occurs as described herein is, in general, the tRNA anticodon stem-loop structure, and most preferably the loop structure itself. Following conventional tRNA nucleotide numbering (see, e.g., M. Sprinzl et al., Compilation of tRNA sequences and sequences of tRNA genes, Nucleic Acids Res. 26, 148-153 (1998)), the site to which binding occurs is from nucleotide 27 or 32 to nucleotide 39, 41 or 43. Nucleotides 32, 34, 35, 37 and 39 are preferred binding sites, and nucleotides 34 and 37 are particularly preferred binding sites. Binding may be to a single site or combination of sites comprising nucleotides within this range.

As noted above, a method of screening for compounds useful for inhibiting S. aureus propagation is disclosed herein. The method involves contacting a specific S. aureus tRNA, such as a specific tRNA^{arg}, to a ribosome that binds that tRNA in the presence of the test compound. The contacting step is typically carried out in vitro in an aqueous solution, with the tRNA, the ribosome, an appropriate messenger RNA, and the test compound in the aqueous solution. The contacting step may be carried out with a single test compound or with a library of probes or test compounds in any of a variety of combinatorial chemistry systems, as discussed in greater detail below.

After the contacting step, the next step involves determining whether the compound inhibits the binding of the specific tRNA to the ribosome (e.g., the binding of tRNA^{arg} at the appropriate position(s) on the ribosome for incorporation of an arginine into a growing peptide or protein.

The determining step can be carried out by any suitable means, such as the filter binding assays disclosed below, or in any of the binding detection mechanisms commonly employed with combinatorial libraries of probes or test compounds as discussed below. Inhibition of ribosomal binding by the test compound indicates that the test compound is useful for inhibiting Staphylococcus aureus propagation. Compounds identified by this technique are sometimes referred to as “active compounds” herein. The method is particularly useful for identifying compounds that inhibit S. aureus growth, preferably bacteria that contain a single tRNA for a particular amino acid, such as a single arginine tRNA that is specific for S. aureus over other bacteria.

A method of screening for compounds useful for inhibiting S. aureus propagation in a host is also disclosed herein. The method comprises contacting the specific host tRNA to the S. aureus RNA in the presence of the test compound. The contacting step is typically carried out in vitro in an aqueous solution, with the tRNA, the S. aureus RNA, and the test compound in the aqueous solution. The term “S. aureus RNA” is intended to encompass both a complete S. aureus genome and fragments thereof that contain the tRNA binding portions (such fragments will typically be at least 10 or 12 to 50 or more nucleotides in length). The contacting step may again be carried out with a single test compound or with a library of probes or test compounds in any of a variety of combinatorial chemistry systems, as discussed in greater detail below.

After the contacting step, the next step involves determining whether the compound inhibits the binding of the specific host tRNA to the S. aureus RNA in the presence of the test compound. The determining step can be carried out by any suitable means, such as gel shift assays, chemical and enzymatic footprinting, circular dichroism and NMR spectroscopy, equilibrium dialysis, or in any of the binding detection mechanisms commonly employed with combinatorial libraries of probes or test compounds as discussed below. The inhibition of binding indicates that the test compound is useful for inhibiting propagation of the S. aureus in the host. Such compounds are also sometimes referred to as “active compounds” herein. The method may be carried out with S. aureus. In one embodiment the specific host tRNA is mammalian, preferably primate or specifically human, such as tRNA^{arg}_{SUU}, and the determining step comprises determining whether the compound inhibits the binding of tRNA^{arg}_{SUU} to the S. aureus RNA.

As noted above, the present invention can be used with test compounds (or “probe molecules”), or libraries (where groups of different probe molecules are employed), of any type. In general, such probe molecules (including those that are active compounds herein) are organic compounds, including oligomers such as antisense olionuleotides, non-oligomers, organo-metallic compounds, and combinations thereof, as well as bio-inorganic compounds. Non-oligomers include a wide variety of organic molecules, such as heterocyclics, aromatics, alicyclics, aliphatics and combinations thereof, comprising steroids, antibiotics, enzyme inhibitors, ligands, hormones, drugs, alkaloids, opioids, benzodiazepenes, terpenes, prophyrins, toxins, and combinations thereof. Oligomers include peptides (that is, oligopeptides) and proteins, oligonucleotides such as DNA, RNA and their derivatives such as peptide nucleic acid (PNA), oligosaccharides, polylipids, polyester, polyamides, polyurethans, polyureas, polyethers, poly(phosphorus derivatives) such ass phosphates, phosphonates, phosphoramides, phosphonamides, phosphites, phosphinamides, etc., poly(sulfur derivatives) such as sulfones, sulfonates, sulfites, sulfonamides, sulfenamides, etc., where for the phosphorous and sulfur derivatives the indicated heteroatom for the most part will be bonded to C, H, N, O or S, and combinations thereof. Numerous methods of synthesizing or applying such probe molecules on solid supports (where the probe molecules may be either covalently or non-covalently bound to the solid support) are known, and such probe molecules can be made in accordance with procedures known to those skilled in the art. See, e.g., U.S. Pat. No. 5,565,324 to Still et al., U.S. Pat. No. 5,284,514 to Ellman et al., U.S. Pat. No. 5,445,934 to Fodor et al. (the disclosures of all United States patents cited herein are to be incorporated herein by reference in their entirety); J. Baldwin and I. Henderson, Recent Advances in the Generation of Small-Molecule Combinatorial Libraries: Encoded Split Synthesis and Solid-Phase Synthetic Methodology, Med. Res. Reviews 16, 391-405 (1996).

Such probe molecules or active compounds could be used as inhibitors by contacting the tRNA, the RNA to which the tRNA binds (mRNA, ribosomal RNA) or the modification enzyme responsible for the unique or unusual chemistry or structure of the tRNA (i.e., the modified base).

I. Methods for Identifying an Inhibitor of S. aureus Propogation

Inhibitors of S. aureus propagation can be identified using the methods described herein. The S. aureus propagation can be inhibited, for example, by inhibiting S. aureus translation of RNA to proteins.

The theory behind the methods for identifying inhibitors of S. aureus propagation, and a way to carry out the method, are discussed below.

Identifying Inhibitors of S. aureus Protein Translation

In one aspect, the method can be used to identify inhibitors of S. aureus translation/protein expression. In another aspect, the methods can be used to identify inhibitors of tRNA binding to a target nucleic acid molecule. In another aspect, the methods can be readily adapted for use in high through-put assays. Transfer RNA (tRNA) is involved in translation through the recognition of a corresponding site on the S. aureus genome priming translation. Identifying inhibitors of translation/protein expression may lead to the identification of therapeutic compounds for use in treating S. aureus infection in a host cell and organism.

The method comprises forming a mixture having a tRNA anticodon stem-loop (ASL) fragment, a target nucleic acid molecule that is capable of binding to the tRNA fragment, and a test compound. In one aspect, the target nucleic acid molecule corresponds to a fragment of the S. aureus genome involved in translation, specifically, involved in incorporation of arginine residues, and, more specifically, incorporation of arginine residues using a tRNA specific for S. aureus, into a protein or peptide necessary for the bacteria to survive.

The resulting mixture is incubated under conditions that allow binding of the tRNA fragment and the target nucleic acid in the absence of the test compound. The method further involves detecting whether the test compound inhibits the binding of the tRNA fragment to the target nucleic acid, where the absence of binding of the tRNA ASL fragment and the target nucleic acid molecule is indicative of the test compound being an inhibitor of S. aureus propogation. In one aspect, the detection involves the use of labels to detect the inhibition of binding of the tRNA fragment to the target nucleic acid molecule.

Protein Synthesis

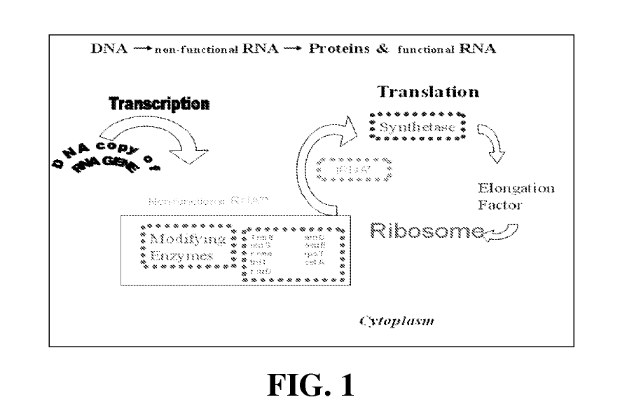

One pathway ideally suited for novel antibiotic discovery is protein synthesis, which involves the ribosome and several types of enzymes. Both the ribosome and the enzymes bind to specific RNA sequences (FIG. 1). What is lacking for the discovery of new classes of antibiotic compounds that target protein synthesis at the ribosome is a method by which to screen large numbers of compounds that may interfere with these RNA interactions. Ashraf (1999), Phelps (2004) and others have discovered that the ribosome:RNA interactions occur at a much higher frequency and with greater affinity in regions of the RNA that contain modified nucleotide bases.

As shown in FIG. 1, protein synthesis requires the involvement of nonfunctional RNA. The nonfunctional RNA(**) which is a substrate of the modifying enzyme is converted to functional RNA either by modifying one or more nucleotide bases. The anticodon stem loop (ASL) of tRNA(*) that contains modified nucleotide bases interacts with the ribosome to transfer specific amino acids to the on-going protein synthesis process.

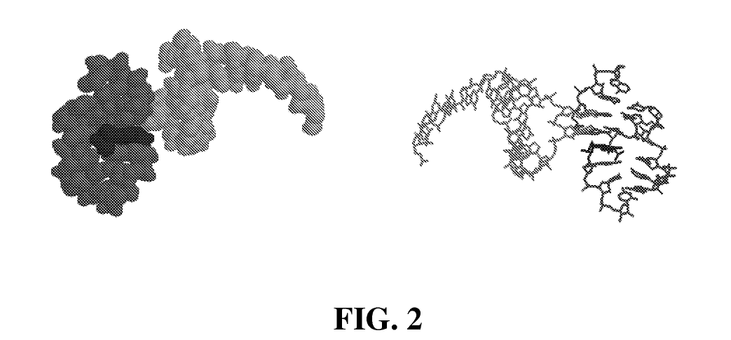

Recent crystallographic investigations illustrate that the post-transcriptional modifications of some tRNAs play an essential role in tRNA recognition by the ribosome translocation site (Phelps et. al. 2004, FIG. 2). These crystallographic studies of the ribosome with ASLs have revealed that the basis for tRNA recognition is a specific group of modified ribosomal residues (FIG. 2).

In addition, binding studies utilizing synthetic RNA oligomers representing the ASL that contains various modified nucleotide bases have also demonstrated an increased affinity of the ribosome with the oligomer containing the modified nucleotide base(s) over the RNA oligomers with unmodified nucleotide bases.

The Role of Modified Nucleotides in Translation

The academic research in the labs of Dr. Paul Agris, NC State University, and Dr. Andrzej Malkiewicz, of the Technical University of Lodz, Poland has focused on understanding a narrow area of RNA biology (See, for example, U.S. Pat. No. 6,461,815). Their research has focused on understanding the role of the natural post-transcriptional modifications in RNA structure and function (Agris et. al. 2004). These modifications are enzyme catalyzed and can be as simple as the addition of a methyl group or they can be quite complex involving a multi-enzyme process (FIG. ).

The details of tRNA^{lys} binding and the critical role of tRNA modifications have been determined at the ribosomal translocation site. FIG. 2 is a recently resolved structure of a tRNA^{Lys} ASL bound to the ribosome at the translocation site of Thermus thermophilus (Murphy et. al., 2004). The atomic resolution structure provides evidence that the basis for increased binding of the native modified tRNA^{Lys} ASL compared to an unmodified ASL is specific atomic interactions with the modified base. While the structural details are less understood, modifications to the nucleotide bases in the anticodon stem loop of tRNA significantly increase the affinity of tRNA to the ribosome at both A and P binding sites (Ashraf et. al. 1999).

Over 100 different naturally occurring modified nucleotides are found in all classes of RNA and all kingdoms of organisms (Limbach et. al. 1994). One to two percent of all RNA nucleotides are modified. These nucleotide base modifications are frequently found near catalytic sites of RNAs and many of the proteins that are responsible for the modification are encoded by essential genes (Zhang 2004). In FIG. , the modified portions of some example nucleotide bases are circled along with the corresponding site on different amino acids to highlight the area of interaction and increased chemical affinity between the nucleotide base and the amino acid. The researchers in Agris and Malkiewicz labs have taken an approach to study model systems produced by chemical synthesis methods rather than using modified nucleotide bases obtained from biochemical methods (Agris et. al. 1995). This synthetic approach provides far more control to investigate, in detail, the significant contributions made by modified nucleotides. Studies using synthetic nucleotide bases have demonstrated the essential role that RNA modifications have in binding at the ribosome as well as in protein synthesis (Hermann, 2005; Francois, et. al. 2005). This approach has also been used in biophysical studies to determine the thermodynamic contribution of modifications (Agris et. al. 1999). In structural studies, this synthetic approach has been able to demonstrate the role of the modified nucleotide basis in producing new structures which are critical in functional capacities (Agris, et. al. 1997).

Synthesis of Modified Nucleotides and Oligomers:

As noted above, a key advancement in elucidating the role and importance of post-transcriptional modification in tRNA binding to the ribosome is the development of synthetic approaches to produce tRNA mimics (Agris, et. al. 1995). The first step in producing the synthetic tRNA mimics is the synthesis of the modified nucleotide bases, also known as phosphoramidites (Agris et al 1995). The modified bases are then used during the synthesis of the RNA oligomers (Ogilvie et. al. 1988). Synthetic approaches overcome the substantial barrier of obtaining sufficient amounts of natural products for the functional characterization studies. In addition to providing the fully modified ASL for characterization of the tRNA:ribosome binding, the synthetic approach allows for the preparation of intermediate forms of the modified material that can further elucidate the individual contribution of each modification step in enhanced tRNA binding. These mimics have been used to demonstrate that the nucleotide modifications to the anticodon increase the affinity of the tRNA for the ribosome by three orders of magnitude (Ashraf, et. al. 1999; Preliminary Data Section 4).

An additional constraint in exploiting these post translational nucleotide base modifications as potential targets is the lack of methods to efficiently synthesize the modified RNA oligomers which incorporate the modified nucleotide bases. This research group has discovered and/or licensed the necessary technology to allow for the synthetic preparation of significant quantities of these natural products (Agris et. al. 1995). In combination with commercially available standard bases, protocols have been developed to allow for the incorporation of hyper-modified nucleotides (phosphoramidites) into oligomers in quantities sufficient to use in RNA based screening assays.

With the oligomers containing modified nucleotide bases, Trana Discovery in conjunction with the technology patent holders has conducted preliminary experiments related to some of the key components required for the development of these assays. These preliminary experiments validate this concept for the discovery of inhibitors with pharmaceutical potential. The next logical step is to develop this concept into an HTS screening assay; thus, this application is focused on the development of an HTS screening assay to identify small molecule inhibitors for development of antibiotics. Specifically, these inhibitors interfere the binding of the tRNA with the ribosome during translation (FIG. )

Characterization of tRNA Binding to the Ribosome In Vitro.

Several laboratories have demonstrated that the binding of tRNA to programmed ribosomes can be replicated in vitro (von Ahsen 1997 Ashraf 1999). Schilling-Bartetzko et. al. (1992) discovered that ribosomes could be purified and programmed with a message and that tRNA would bind to various sites on the ribosome based on the solution conditions. These binding reactions are currently performed as individual reactions with the ASL:ribosome complex being bound to filter papers. In addition to these reactions being conducted in a large volume, they use radioactive materials for detection and quantitation. While these methods do provide an approach to characterize tRNA:ribosome binding they are not compatible with HTS assays due to the size of the reaction vessel, the radioactive detection methodology, and the subsequent radioactive waste disposal. A fluorescent method of detection to monitor tRNA binding to the ribosome has been developed (Wells et. al. 1980) that can be adapted to an HTS format.

Preliminary Data:

Combining all of the facts from above (translation as a biochemical target; ASL of tRNA containing modified nucleotides are essential to translation; that one can synthesize the modified bases and RNA ologimers containing these modified bases; and, that these oligomers bind to programmed ribosomes), the present inventors have conducted a series of experiments to demonstrate that a fluorescently labeled synthetic oligomer containing 17 nucelotide bases, 3 of which are modified, (FIG. ) will bind to programmed ribosomes isolated from S. aureus, and that this binding can be detected by monitoring the change in fluorescence.

In moving from a manual radioactive filter based assay to a format suitable for adaptation to a HTS format, efforts were focused in two objectives. Concurrent efforts were made on the preparation of the phosphoramidite components for the synthesis of a modified oligonucleotide (FIG. ) while experiments were performed to adapt the assay format.

Developing high throughput assays to identify compounds that inhibit RNA:ribosome interactions is the basis of this application. The development of this HTS assay requires several components including: selection and synthesis of the substrates (oligomers which contain modified nucleotides); detection methodologies; conversion of standard methods to high throughput methods; and, proof that these RNA oligomers will serve as ribosome substrate in an HTS assay. Preliminary data to support the selection of the RNA oligomer to use as a substrate for this assay, the assay format (filter paper vs. in solution), and for the assay conditions were generated by Trana scientists. As described in the following paragraphs Trana scientists have obtained preliminary data to support: (1) the selection of the RNA oligomer to use as a substrate for this assay; (2) the assay format (filter paper vs. in solution), and (3) the assay conditions.

Selection of Test Sequence.

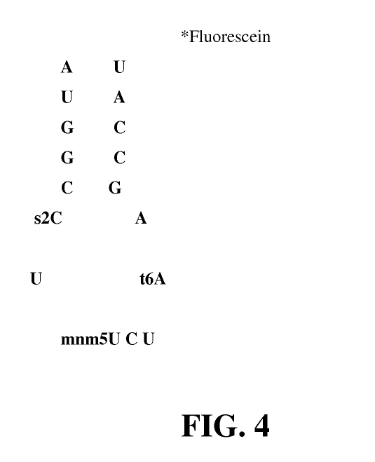

The initial survey of the modification requirement for an ASL to bind to the ribosome (Yarian 2002) identified several different ASL where the unmodified oligomer poorly bound to programmed ribosomes. These ASLs become good candidates for tools to be used to screen for compounds that can block selected tRNA from binding to the ribosome and selectively inhibit bacterial protein synthesis. Based on the phosphoramidites available for oligonucleotide synthesis tRNA^{Arg}_{UCU} was selected. Using the complete genome for S. aureus the sequences of tRNA^{Arg}_{UCU} were surveyed and a single sequence was found for the multiple copies in the genome, FIG. . FIG. 4 is an illustration of a synthetic oligomer representing the anticodon stem loop from tRNA^{Arg} in S. aureus. Only when the modifications are present will the ASL bind to programmed ribosomes isolated from S. aureus, which binding can be detected by monitoring the change in fluorescence. The synthesis of the AS^{Arg}_{UCU} has been completed and the correctness of the composition was confirmed by mass spectrometry analysis.

Synthesis of Modified Nucleotide Base(s) and RNA Oligomers:

Using a combination of methods from the literature and proprietary information, the present inventors have developed protocols for the synthesis, incorporation and purification of all the modified nucleotides found in the tRNA^{Arg} ASL from S. aureus (Agris et. al. 1995, Ashraff, 1999, unpublished data 2007).

As previously shown in FIG. , the tRNA^{Arg} ASL contains 1 modified base denoted as t6A which was prepared following the previously published procedures of Ryszard et. al. and Sundaram, et. al. (FIG. ). The tRNA^{Arg} also contains 2 modified nucleotides, s2C and mnm5U, for which synthesis methods were developed by the present inventors.

In general, functional groups on the modified nucleotide bases are protected using phosphoramidite chemistry (Ogilvie et. al. 1988). Using this chemistry, the founders have incorporated over 20 different modified nucleotides into a range of oligonucleotides ranging in length from 3 to 36 nucleotides (Nobles et. al. 2002). The protecting groups were subsequently removed after synthesis of the RNA oligomer. The addition of a protecting group to each modified base and ribose is described below and shown in FIG. . Because the 2 position thio-groups in the modified RNA nucleotides can be oxidized in standard RNA synthesis an alternative oxidizing agent, tert-butyl hydroperoxide (10% solution in acetonitrile) (Kumar and Davis, 1997), was used during synthesis of the oligomer along with other proprietary techniques. The founders have used these synthetic RNA oligomers in both functional (Yarian 2000, 2002 and Phelps 2004) and structural studies (Stuart 2000 and Murphy 2004).

Modified Nucleotide t6A Phosphoramidite

Preparation of the N6-(N-threonylcarbonyl) adenosine for automated synthesis followed a slightly different approach than that for the other phosphoramidites (FIG. Panels A, B, and C). The first step was to protect the ribose functions of adenosine. Next, the ribose protected adenosine was reacted with 3 equivalents of phenoxycarbonyltetrazole in anhydrous dioxane for 18 hr at 37° C. to produce phenyl carbamates at the six position. This was followed by aminolysis with 3 equivalents of crystalline L-threonine p-nitrobenzyl ester in anhydrous dioxane, for 18 hr at 37° C., producing the N6-(N-threonylcarbonyl) adenosine. The t6A carboxylate was then protected by a trimethylsilylethyl group, in a manner similar to that used to protect the ribose function. Finally the phosphoramidite was phosphitylated following the protocol described above.

Modified Nucleotides s2C and mnm5U

These modified nucleotides were synthesized by the present inventors using proprietary methods. In general, the exo-amino function of the core nucleotide base, C or U, was protected following a scheme similar to Malkiewicz (Malkiewicz et. al. 1983). Following protection of the nucleotide base, the ribose function was then protected and phosphitylated using these general procedures (Panel C, FIG. ). The protected nucleotide was dried by co-evaporation twice with pyridine and dissolved in pyridine. Tert-butyldimethylchlorosilane and imidazole were added and reacted for 4 hours at room temperature. The excess silyl chloride was decomposed with water and dichloromethane. The aqueous layer was extracted twice with dichloromethane and combined with the organic layer. The solvent was evaporated by vacuum yielding a gum which was dissolved in ether and precipitated by pouring slowly into petroleum ether (40-60° C.) with stirring. The precipitate was collected and washed twice with petroleum ether. At this point the crude product contained three components; the 2′,3′ disilylated, 2′ silylated (major product) and 3′ silylated. The pure 2′ protected isomer was obtained by silica gel column chromatography. This product was then ready for phosphitylation.

The N-protected-5′-O-dimethoxytrityl-2′-O-tertbutyldimethylsilyl-ribonucleotides were dried by two co-evaporations with anhydrous pyridine and THF. The residue was dissolved in anhydrous THF under argon. Dimethylaminopyridine, N,N,N-ethyidiisopropylamine and cyano-ethoxydiisopropy amino-chlorophosphine were added through a rubber septum. After 2 hours the reaction mixture, was quenched with ethyl acetate and washed with 5% sodium bicarbonate followed by water. Aqueous washes were back extracted with ethyl acetate. Combined organic layers were dried over sodium sulphate and the solvent was evaporated yielding viscous oil. The product was co-evaporated twice with toluene and the pale yellow phosphoramidite products were purified by flash silica gel chromatography.

Synthesis of RNA Oligomers Containing Modified Nucleotide Bases:

The RNA oligomer was synthesized and purified following protocols developed specifically for these modified reagents (Agris et. al. 1995, Murphy et. al. 2004). Purification of the oligomers was by HPLC as previously described (Agris et. al., 1999). Purity of the oligomer was confirmed by gel electrophoresis and proper incorporation of the modified nucleotide bases was confirmed by mass spectrometry.

In addition to the ASL^{Arg} containing the 3 modified nucleotide bases, ASL^{Arg} containing no modified bases and a random 17 mer oligomer were synthesized to be used to demonstrate specificity and as a negative control, respectively. All oligomers were tagged with fluorescein on the 3′ end.

In general the synthesis of an RNA oligomer requires that all of the major functional groups on each nucleotide base be protected during the formation of the oligomer and then deprotected after synthesis. The general protection, deprotection and oligomer synthesis schemes developed by the company founders are described in the following paragraphs.

Procedures of the Deprotection of Synthetic Oligoribonucleotides (RNA Oligos)

Several protecting groups are available and are selected based upon the specific chemistry of each nucleotide base (FIG. ); thus, the protection group on the RNA phosphoramidite monomers to a large extent will dictate the strategy for deprotection. It is routine in the art to remove silyl protecting groups with tetrabutylammonium fluoride solution and triethyamine trihydrifluoride.

For regular deprotection of the phosphoramidite protecting groups, ethanolic ammonium hydroxide is added to the vial containing the beads from the synthesis process and incubated—time specific to the types of protecting groups. For removal of the Silyl protecting groups on the sugars, tetrabutylammonium fluoride solution is added to the residue from deprotection step.

The 4 steps described in the following paragraphs are required to add each nucleotide to the oligomer are illustrated in FIG. .

Step A: De-blocking

The first base, which is attached to the solid support, is at first inactive because all the active sites have been blocked or protected. To add the next base, the DMT group protecting the 5′-hydroxyl group must be removed. This is done by adding a base, either dichloroacetic acid (DCA) or trichloroacetic acid in dichloromethane (DCM), to the reaction column. The 5′-hydroxyl group is now the only reactive group on the base monomer. This ensures that the addition of the next base will only bind to that site. The reaction column is then washed to remove any extra acid and by-products.

Step B: Base Condensation

The next base monomer cannot be added until it has been activated. This is achieved by adding tetrazole to the base. Tetrazole cleaves off one of the groups protecting the phosphorus linkage. This base is then added to the reaction column. The active 5′-hydroxyl group of the preceding base and the newly activated phosphorus bind to loosely join the two bases together. This forms an unstable phosphite linkage. The reaction column is then washed to remove any extra tetrazole, unbound base and by-products.

Step C: Capping

When the activated base is added to the reaction column some does not bind to the active 5′-hydroxyl site of the previous base. If this group is left unreacted in a step it is possible for it to react in later additions of different bases. This would result in an oligonucleotide with a deletion—and an incorrect sequence manufactured. To prevent this from occurring, the unbound, active 5′-hydroxyl group is capped with a protective group which subsequently prohibits that strand from growing again. This is done by adding acetic anhydride and N-methylimidazole to the reaction column. These compounds only react with the 5′-hydroxyl group. The base is capped by undergoing acetylation. The reaction column is then washed to remove any extra acetic anhydride or N-methylimidazole.

Step D: Oxidation

In step 2 the next desired base was added to the previous base, which resulted in an unstable phosphite linkage. To stabilize this linkage a solution of dilute iodine in water, pyridine, and tetrahydrofuran (when synthesizing DNA) is added to the reaction column. For RNA syntheses, see previous paragraphs describing our techniques for consideration made at this step. The unstable phosphite linkage is oxidized to form a much more stable phosphate linkage.

Repeat above steps for entire sequence being synthesized.

Steps one through four are repeated until all desired bases have been added to the oligonucleotide. Each cycle is approximately 94/95% efficient in unmodified RNA and approximately 91/92% efficient in highly modified units of RNA phosphoramidites incorporated into oligonucleotides.

Post Synthesis Treatment to Remove Protection Groups

After all bases have been added, the oligonucleotide must be cleaved from the solid support and deprotected before it can be effectively used. This is done by incubating the chain in appropriate solutions previously described. Once all the protecting groups are cleaved, including the cyanoethyl group, the heterocyclic protection groups, and the DMT group, the oligonucleotide will be functional and ready to use (FIG. ).

S. aureus Ribosome Assay Development:

The assay development experiments initially used unmodified 17 nucleotide control ASLs complementary to E. coli tRNA^{Phe} which are able to bind to ribosomes programmed with a poly U message. The initial control sequences tested both a 3′ and 5′ fluorescein to determine if location of the fluorophore affected the limit of detection or affinity of the ASL to the ribosome. Titrations of the fluorescent ASL under a range of conditions were made to determine that the limit of detection was about 3×10⁻⁷M which is suitable for the needs of the assay.

This preliminary data was used to determine starting conditions to test if the existing filter disk protocol could be modified to a 96 well plate assay, such as a 96 well filtration plate assay, using a Millipore 96 well HTS filter plate. By slightly modifying the conditions used in the filter assay message dependent ASL binding was observed and a comparable tRNA ribosome binding activity obtained for the whole tRNA in a radiochemical activity. To convert the assay from the filter based assay format which is labor intensive and has low throughput to a solution based assay that is more compatible with HTS format, efforts were shifted to testing if ASL binding could be detected in solution using fluorescence. Using buffer conditions similar to the initial binding steps in the filter assay, addition of the ASL to the programmed ribosome resulted in a quenching of the ASL fluorescence in a specific manner. These results indicate that the oligomer is binding to the programmed ribosome and that this binding is detectable by monitoring the change in fluorescence.

During translation, ribosomes are programmed to accept tRNA carrying the appropriate amino acid. For this project, ribosomes are programmed to receive the tRNA associated with ASL^{Arg} using the message, poly AGA. The binding affinity of correctly modified ASL to ribosomes programmed with short messages is comparable to those observed for native tRNA and full message (Ashraf et. al. 1999); thus, confirming that the entire tRNA is not required for effective ribosomal binding.

Thus, a series of experiments were conducted: i) to demonstrate that the synthetic RNA oligomer containing the 3 modified nucleotide bases described above will bind to programmed ribosomes using the historical filter paper technique and that this binding could be monitored using fluorescent detection in a 96-well plate format; ii) to demonstrate specificity of the programmed ribosomes to the ASL^{Arg}; iii) to demonstrate that this filter based assay could be converted to an HST compatible solution based assay by monitoring the differential fluorescent signal associated with bound vs. unbound ASL^{Arg}; and, iv) to determine that an acceptable limit of detection could be achieved with the solution based assay. The results from these experiments are the basis for the research program required to convert this into a validated HTS assay.

Preliminary work focused on determining the limit of detection and to confirm that only minimal non-specific retention of ASL by the filter manifold occurred. Titrations of the fluorescent ASL under a range of conditions were made to determine that the limit of detection was about 3×10⁻⁷M establishing an acceptable working concentration range for the fluorescently labeled ASL. These data were used to determine starting conditions for the assay. Using a 96-well filtration plate assembly in conjunction with a Millipore 96 well HTS filter plate, message dependent ASL binding was observed (FIG. ), i.e. binding of the fluorescently labeled RNA oligomer increased significantly in programmed vs. unprogrammed ribosomes. These results are comparable to tRNA ribosome binding activity obtained for the whole tRNA in a radiochemical activity assay.

This basic assay will detect binding of the RNA oligomer to programmed ribosomes, and the programmed ribosomes are specific to the ASL^{Arg} oligomer synthesized for this project.

Use of Combinatorial Chemistry to Identify and Optimize Leads

Mechanistically and biologically active hits can be identified using compound libraries, such as lead generation libraries, i.e., libraries including between around 10 and around 500,000 compounds. This risk can be minimized by selecting a diverse library; however, subsequent screening of another library is also a possibility. The assay can be used to identify active compounds that are specific to S. aureus.

As presented in the preliminary data, the substrates (RNA oligomer containing modified nucleotide bases and S. aureus ribosomes) to be used in this assay have been synthesized.

Once leads are identified, mammalian cell toxicity testing can be conducted, and computational modeling in SA6 can also be conducted.

The assay can be further developed and optimized for automation, or can be run manually. The manual assay can be conducted, for example, in 96-well plate format. The conversion of this assay to HTS format typically involves reducing the volumes of the kit components for use in a 384 or possibly 1,536 plate format, and optimizing the conditions for the capabilities available with a given robotic system. For example, during the early phase of development of the manual assay, the assay mixture was incubated at 37° C. following previous protocols; however, incubating at this temperature is difficult to accomplish in some facilities. To compensate for this, preliminary experiments were conducted to determine that incubation at 25° C., but for a longer period of time.

When the volume of all substrates is reduced, the amount of the fluorescent label is also reduced; thus, reducing the amplitude of the signal to be detected. This is generally overcome by using detectors specifically developed for HTS assays or using other means to increase the signal differential between bound vs. unbound substrate. To ascertain the effect of the assay reagents and/or assay conditions on assay performance, each condition can be varied within predetermined ranges and the assay results will be analyzed as described below. Assay conditions can be modified, for example, until a Z-factor between 0.4 and 1 is obtained (see next paragraph). Z-factors are an industry standard method for determining when an assay has been optimized.

To calculate the Z-factor, data from the optimization experiments will be recorded electronically, stored to disk, and directly imported into an electronic spreadsheet for analysis. Data will be analyzed to determine the assay value ratio (AVR) defined as 3(Sp+Sb)/(Xt−Xb), where: Sp and Sb are standard deviation of positive control signal and background signal; and, Xp and Xb are the averages of the positive control totals and backgrounds (Zhang, et. al. 1999). A value of less than 0.6 indicates suboptimal assay performance. The Z factor is determined as (1-AVR); therefore, the target value is between 0.4 and 1.0.

The HTS assay can be validated, for example, by analyzing a suitable library (for example, including approximately 6,000 compounds including positive and negative controls) in duplicate using the HTS robotic systems.

An industry standard diverse chemical library (eg., Preswick library) supplemented in random order with positive and negative controls along with other selected chemicals can be used in this validation experiment. During the first run, this validation will demonstrate that the positive and negative controls can be determined in a consistent manner and that a range of activity is detected in the remaining compounds. This will demonstrate that the assay is robust and functioning properly on the complete robotic system.

Typically, if the assay is determined not to be robust, then some mechanical aspect of the robotic system needs to be evaluated. For example, the stability of reagents and substrates in the tubing and mechanical portions of the robotic systems may be the cause of the inconsistent results. These challenges are generally not insurmountable, just time consuming and causing delays in the project. In some cases if the results are totally unacceptable, then the first day results are discarded and this experiment is rerun after identifying and fixing the item(s) that caused the inconsistent results. After the first day experiment is completed with acceptable results, the experiment is repeated on a second day simply to confirm the results from the first experiment.

In addition to the positive and negative controls, the validation library contains compounds that are generally known to be general toxins that will inhibit most assays. Another criterion for assay validation is the ability of the assay to identify these general toxins while not being inhibited by all compounds in the library.

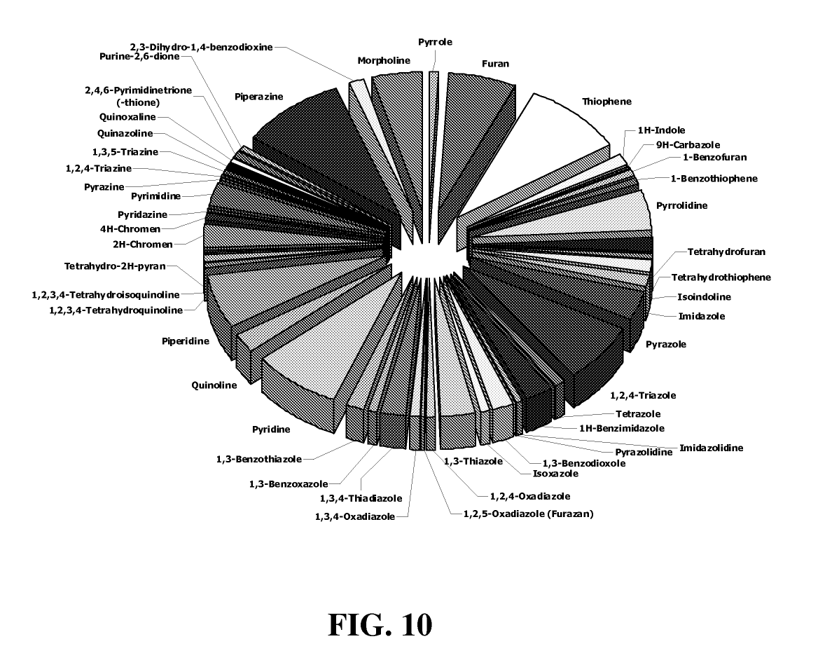

A diverse chemical library can be screened using the validated assay. As used herein, the term library refers to 100 or more compounds, ideally, 10,000 or more compounds, and preferably, includes a minimum of 100,000 compounds or more. A subset of a diverse chemical library will be screened with the validated assay at a single concentration. Ideally, diverse and targeted libraries will total approximately 500,000 compounds, consisting of a mix of structurally diverse singletons and compound clusters. The clusters are ideally built around a variety of scaffolds, each class containing enough members to provide a preliminary SAR analysis if the entire cluster is screened. The libraries ideally include a modest selection of natural products and known drugs, though natural products were not emphasized during compound acquisition because of their frequent synthetic intractability. The collection has been characterized for diversity (for example, by Tanimoto coefficients, as implemented in the Selector software module of the SYBYL modeling package), and individual members have been characterized for biological relevance by, for example, their Lipinski parameters and molecular fingerprints known to favor small molecule-protein interactions. The anticipated distribution of one 100 k subset, which is a representative compound library, is depicted in FIG. 10. Portions of this library have been screened in over 80 assays with over 16,000 hits confirmed in dose response studies.

Depending upon the throughput of the validated assay (384 vs. 1,536 plate format), this typically takes one or two days on a robotic system, and approximately one week for data analysis. In a typical screening campaign, 3 to 5% of the compounds are expected to demonstrate some level of activity. Those compounds that demonstrate sufficient inhibitory activity in the assay can be re-screened in a dilution series (5 to 10 concentrations) to confirm that the compounds are inhibitory and to establish IC₅₀ and IC₉₀ concentrations. A range of inhibitory activity is typically observed in these experiments. Generally, those compounds referred to as ‘hits’ that demonstrate the strongest inhibitory effects (lower inhibitory concentrations) are selected for advancement to the next step, for example, subsequent screening in biological assays as described herein.

In some cases, a manual review of the chemical structures of the compounds demonstrating inhibitory activity will be conducted by an expert in structure-activity-relationships, and compounds that are less inhibitory will also be selected for further screening based on favorable chemical structure characteristics.

Confirming the Biological Activity of Hits from the HTS

The biological activity of the identified ‘hits’ from the screening assays described above can be determined by analyzing these hits in a minimum inhibitory compound bacterial screen.

Screening of the ‘hits’ for antimicrobial activity to determine 1) a single breakpoint concentration of activity against a common Gram-positive and a Gram-negative human pathogen; and 2) whether a “clinically significant” potency can be detected using a threshold concentration of 32 μg/ml. For determining the single breakpoint of activity, a well-characterized strain of Escherichia coli American Type Culture Collection (ATCC) 25922 and Staphylococcus aureus ATCC 29213 will be tested against each sample. As a minimum inhibitory concentration (MIC), an achievable potency of less than or equal to 32 μg/ml would be considered an active sample worthy of extended study.

The process will use the reference broth microdilution methods recommended by the Clinical and Laboratory Standards Institute (CLSI; formerly the National Committee for Clinical Laboratory Standards [NCCLS]) M7-A7 document. A 96 well microtiter tray assay will be used to test a single concentration of 32 μg/ml against each pathogen species. A working concentration of 64 μg/ml will be made using appropriate solvents and diluents. A calibrated pipette will be used to transfer 50 μL of each sample into one well of each of three 96-well microtitre plates. A standard inoculum (equivalent to a 0.5 MacFarland standard) of each microorganism will be made in Mueller-Hinton broth and 50 μL of a diluted sample to achieve 3-5×10⁵ CFU/ml will be added to each sample diluting the sample 1:1 to a final test concentration of 32 μg/ml. Each batch of samples will include two internal quality controls using antimicrobial agents with a known potency range and targeting protein syntheses. A positive growth control with only growth support media and an ethanol control at concentrations equivalent to that in the samples will also be tested for each pathogen. After the broth microdilution plates are inoculated, they are incubated in an ambient air environment at 35° C. for 20-24 hours. The plates are removed from incubation and each well is inspected for growth. If a well is clear of growth (non-turbid), an MIC of ≦32 μg/ml is achieved and the sample is defined as an active sample and subject to further investigation.

Those compounds that are determined to be active at a single concentration will then be tested in a dilution series against the same organisms. Those compounds that are the most active will be advance to the secondary screen described in the next paragraph. The conduct of these first two screens on a limited number of species will allow for a greater number of compounds active at the molecular level to be tested at the whole organism level increasing the opportunity to identify biologically active compounds in a very cost effective manner.

A secondary screen of “active” samples will include an extended dilution screen (eight to 12 log₂ dilution steps) to determine “on-scale” value for a potential antimicrobial agent including evaluation of breadth of spectrum to such organisms including staphylococci, streptococci, Enterobacteriaceae, non-fermentative Gram-negative bacilli, anaerobes and yeast species. These isolates will be recent clinical strains representing wild-type and strains with resistance phenotypes. Methods utilized will be those described above with the addition of NCCLS M11-A6 (anaerobes) and M7-A2 (yeast). Testing against this broader spectrum of organisms will characterize the spectrum of antimicrobial activity. In one embodiment, the compounds will be effective against S. aureus, but not against other bacterial species.

The conduct of the initial MIC and the secondary antimicrobial screening will follow industry accepted methodologies and carry no risk in regards to the ability to conduct this testing.

Estimating the Mammalian Toxicity of Biologically Active Compounds

The potential mammalian toxicity of biologically active compounds identified in SA assay can be estimated using 3 mammalian cell lines. Compounds that are highly toxic in these assays will be considered to be general toxins with a high probability of interacting with multiple molecular targets. Cytotoxicity assays will be conducted with rat hepatoma H4IIE cell line, rat kidney NRK cell line, and primary human hepatocytes. Each cell line will be exposed to 5 concentrations of each test article or with appropriate positive and negative control substances. Three biochemical endpoints will be monitored to determine viability, mitochondrial function, and membrane integrity.

The conduct of the mammalian cell toxicity carries no risk in regards to the ability to conduct this project; however, there is a slight risk that all compounds will be determined to be toxic to mammalian cells.

The assay described herein can be used to better understand the tRNA ribosome interactions that occur during translation, and used to screen large compound libraries to discover novel antibiotics. The lead compounds identified during the automated HTS can be further developed for the treatment of S. aureus infections. The new antibiotics can reduce the potential for the development of drug resistance and for the treatment of currently-existing multi-drug resistant organisms.

### II. Pharmaceutical Compositions

The S. aureus inhibitors described herein can be incorporated into pharmaceutical compositions and used to treat or prevent a condition or disorder in a subject susceptible to such a condition or disorder, and/or to treat a subject suffering from the condition or disorder. The pharmaceutical compositions comprise an active compound or a pharmaceutically acceptable salt thereof, in any pharmaceutically acceptable carrier.

Optically active compounds can be employed as racemic mixtures, as pure enantiomers, or as compounds of varying enantiomeric purity. The pharmaceutical compositions described herein include the inhibitors and a pharmaceutically acceptable carrier and/or excipient.

The manner in which the compounds are administered can vary. The compositions are preferably administered orally (e.g., in liquid form within a solvent such as an aqueous or non-aqueous liquid, or within a solid carrier). Preferred compositions for oral administration include pills, tablets, capsules, caplets, syrups, and solutions, including hard gelatin capsules and time-release capsules. Compositions may be formulated in unit dose form, or in multiple or subunit doses. Preferred compositions are in liquid or semisolid form. Compositions including a liquid pharmaceutically inert carrier such as water or other pharmaceutically compatible liquids or semisolids may be used. The use of such liquids and semisolids is well known to those of skill in the art.

The compositions can also be administered via injection, i.e., intraveneously, intramuscularly, subcutaneously, intraperitoneally, intraarterially, intrathecally; and intracerebroventricularly. Intravenous administration is a preferred method of injection. Suitable carriers for injection are well known to those of skill in the art, and include 5% dextrose solutions, saline, and phosphate buffered saline. The compounds can also be administered as an infusion or injection (e.g., as a suspension or as an emulsion in a pharmaceutically acceptable liquid or mixture of liquids).

If a solution is desired, water is the carrier of choice with respect to water-soluble compounds or salts. With respect to the water-insoluble compounds or salts, an organic vehicle, such as glycerol, propylene glycol, polyethylene glycol, or mixtures thereof, may be suitable. In the latter instance, the organic vehicle may contain a substantial amount of water. The solution in either instance may then be sterilized in any suitable manner, preferably by filtration through a 0.22 micron filter. Subsequent to sterilization, the solution may be filled into appropriate receptacles, such as depyrogenated glass vials. Of course, the filling should be done by an aseptic method. Sterilized closures may then be placed on the vials and, if desired, the vial contents may be lyophilized.

In addition to active compounds or their salts, the pharmaceutical compositions may contain other additives, such as pH adjusting additives. In particular, useful pH adjusting agents include acids, such as hydrochloric acid, bases or buffers, such as sodium lactate, sodium acetate, sodium phosphate, sodium citrate, sodium borate, or sodium gluconate. Further, the compositions may contain anti-microbial agents. Useful antimicrobial agents include methylparaben, propylparaben, and benzyl alcohol. The microbial preservative is typically employed when the formulation is placed in a vial designed for multidose use. Of course, as indicated, the pharmaceutical compositions of the present invention may be lyophilized using techniques well known in the art.

In yet another aspect of the present invention, there is provided an injectable, stable, sterile composition comprising an active compound or a salt thereof, in a unit dosage form in a sealed container. The compound or salt is provided in the form of a lyophilizate which is capable of being reconstituted with a suitable pharmaceutically acceptable carrier to form a liquid composition suitable for injection thereof into man. The unit dosage form typically comprises from about 10 mg to about 10 grams of the compound or salt. When the compound or salt is substantially water-insoluble, a sufficient amount of emulsifying agent which is physiologically acceptable may be employed in sufficient quantity to emulsify the compound or salt in an aqueous carrier. One such useful emulsifying agent is phosphatidyl choline.

The formulations may also be administered using other means, for example, rectal administration. Formulations useful for rectal administration, such as suppositories, are well known to those of skill in the art. The compounds can also be administered by inhalation (e.g., in the form of an aerosol either nasally or using delivery articles of the type set forth in U.S. Pat. No. 4,922,901 to Brooks et al., the disclosure of which is incorporated herein in its entirety); topically (e.g., in lotion form); or transdermally (e.g., using a transdermal patch, using technology that is commercially available from Novartis and Alza Corporation). Although it is possible to administer the compounds in the form of a bulk active chemical, it is preferred to present each compound in the form of a pharmaceutical composition or formulation for efficient and effective administration.

Other pharmaceutical compositions may be prepared from the active compounds, such as aqueous base emulsions. In such an instance, the composition will contain a sufficient amount of pharmaceutically acceptable emulsifying agent to emulsify the desired amount of the active compound or salt thereof. Particularly useful emulsifying agents include phosphatidyl cholines, and lecithin.

Further, the present invention provides liposomal formulations of the active compounds or salts thereof. The technology for forming liposomal suspensions is well known in the art. When the active compound or salt thereof is an aqueous-soluble salt, using conventional liposome technology, the same may be incorporated into lipid vesicles. In such an instance, due to the water solubility of the compound or salt, the compound or salt will be substantially entrained within the hydrophilic center or core of the liposomes. The lipid layer employed may be of any conventional composition and may either contain cholesterol or may be cholesterol-free. When the compound or salt of interest is water-insoluble, again employing conventional liposome formation technology, the salt may be substantially entrained within the hydrophobic lipid bilayer which forms the structure of the liposome. In either instance, the liposomes which are produced may be reduced in size, as through the use of standard sonication and homogenization techniques.

Another type of delivery is by implantable drug delivery depots, which typically include a hydrophilic biocompatible, and optionally biodegradable polymer with the active agent physically contained within the structure. The active agent is released by its permeation of and diffusion through the polymer or copolymer structure. The depot may be designed to release the substance or substances at predetermined rates and in predetermined sequence. One type of depot system is of the kind disclosed in U.S. Pat. No. 4,450,150, in which the co-polymer is a poly(glutamic acid-co-ethyl glutamate) co-polymer, which ultimately biodegrades to glutamic acid. Other suitable depot based drug delivery vehicles include polyethylene glycol, and copolymers thereof. Among the preferred configurations for the depots are rods and closed-end capsules.

Pharmaceutical formulations are also provided which are suitable for administration as an aerosol, by inhalation. These formulations comprise a solution or suspension of the desired active compound or a salt thereof or a plurality of solid particles of the compound or salt. The desired formulation may be placed in a small chamber and nebulized. Nebulization may be accomplished by compressed air or by ultrasonic energy to form a plurality of liquid droplets or solid particles comprising the compounds or salts. The liquid droplets or solid particles should have a particle size in the range of about 0.5 to about 5 microns. The solid particles can be obtained by processing the compound, or a salt thereof, in any appropriate manner known in the art, such as by micronization. Most preferably, the size of the solid particles or droplets will be from about 1 to about 2 microns. In this respect, commercial nebulizers are available to achieve this purpose.

Preferably, when the pharmaceutical formulation suitable for administration as an aerosol is in the form of a liquid, the formulation will comprise a water-soluble active compound or a salt thereof, in a carrier which comprises water. A surfactant may be present which lowers the surface tension of the formulation sufficiently to result in the formation of droplets within the desired size range when subjected to nebulization.

Exemplary methods for administering such compounds will be apparent to the skilled artisan. The usefulness of these formulations may depend on the particular composition used and the particular subject receiving the treatment. These formulations may contain a liquid carrier that may be oily, aqueous, emulsified or contain certain solvents suitable to the mode of administration.

The compositions can be administered intermittently or at a gradual, continuous, constant or controlled rate to a warm-blooded animal (e.g., a mammal such as a mouse, rat, cat, rabbit, dog, pig, cow, or monkey), but advantageously are administered to a human being. In addition, the time of day and the number of times per day that the pharmaceutical formulation is administered can vary.

Preferably, the compositions are administered such that active ingredients interact with regions where microbial infections are located. The compounds described herein are very potent at treating these microbial infections.

In certain circumstances, the compounds described herein can be employed as part of a pharmaceutical composition with other compounds intended to prevent or treat a particular microbial infection, i.e., combination therapy. In addition to effective amounts of the compounds described herein, the pharmaceutical compositions can also include various other components as additives or adjuncts.

Combination Therapy

The combination therapy may be administered as (a) a single pharmaceutical composition which comprises an inhibitor as described herein, at least one additional pharmaceutical agent described herein, and a pharmaceutically acceptable excipient, diluent, depot, such as a polyethylene glycol depot, or carrier; or (b) two separate pharmaceutical compositions comprising (i) a first composition comprising an inhibitor as described herein and a pharmaceutically acceptable excipient, diluent, depot, or carrier, and (ii) a second composition comprising at least one additional pharmaceutical agent described herein and a pharmaceutically acceptable excipient, diluent, depot, or carrier. The pharmaceutical compositions can be administered simultaneously or sequentially and in any order.

In use in treating or preventing microbial disease, the inhibitors can be administered together with at least one other antimicrobial agent as part of a unitary pharmaceutical composition. Alternatively, it can be administered apart from the other antimicrobial agents. In this embodiment, the inhibitors and the at least one other antimicrobial agent are administered substantially simultaneously, i.e. the compounds are administered at the same time or one after the other, so long as the compounds reach therapeutic levels for a period of time in the blood.

Combination therapy involves administering the inhibitors, as described herein, in combination with at least one anti-microbial agent, ideally one which functions by a different mechanism (i.e., by penetrating the bacterial cell wall, or interfering with one or more receptors and/or enzymes in the bacteria).

Representative Antibacterial Compounds

Examples of antibacterial compounds include, but are not limited to, aminoglycosides, ansamycins, carbacephems, carbapenems, cephalosporins (First, Second, Third, Fourth and Fifth Generation), glycopeptides, macrolides, monobactams, penicillins and beta-lactam antibiotics, quinolones, sulfonamides, and tetracyclines.

Representative aminoglycosides include Amikacin, Gentamicin, Kanamycin, Neomycin, Netilmicin, Streptomycin, Tobramycin, and Paromomycin. Representative ansamycins include Geldanamycin and Herbimycin. These agents function by binding to the bacterial 30S or 50S ribosomal subunit, inhibiting the translocation of the peptidyl-tRNA from the A-site to the P-site and also causing misreading of mRNA, leaving the bacterium unable to synthesize proteins vital to its growth.

Loracarbef is a representative carbacephem. Representative carbapenems include Ertapenem, Doripenem, Imipenem/Cilastatin, and Meropenem.

Representative first generation cephalosporins include Cefadroxil, Cefazolin, Cephalothin, and Cephalexin. Representative second generation cephalosporins include Cefaclor, Cefamandole, Cefoxitin, Cefprozil, and Cefuroxime. Representative third generation cephalosporins include Cefixime, Cefdinir, Cefditoren, Cefoperazone, Cefotaxime, Cefpodoxime, Ceftazidime, Ceftibuten, Ceftizoxime, and Ceftriaxone. Cefepime is a representative fourth generation cephalosporin, and Ceftobiprole is a representative fifth generation cephalosporin.

Representative glycopeptides include Teicoplanin and Vancomycin, which function by inhibiting peptidoglycan synthesis.

Representative macrolides include Azithromycin, Clarithromycin, Dirithromycin, Erythromycin, Roxithromycin, Troleandomycin, Telithromycin, and Spectinomycin, which function by inhibiting bacterial protein biosynthesis by binding irreversibly to the subunit 50S of the bacterial ribosome, thereby inhibiting translocation of peptidyl tRNA.

Aztreonam is a representative monobactam.

Representative penicillins include Amoxicillin, Ampicillin, Azlocillin, Carbenicillin, Cloxacillin, Dicloxacillin, Flucloxacillin, Mezlocillin, Meticillin, Nafcillin, Oxacillin, Penicillin, Piperacillin, and Ticarcillin. These can be administered with an agent which inhibits beta-lactamase enzymatic activity, such as potassium clavanulate or clavulanic acid.

Representative quinolones include Ciprofloxacin, Enoxacin, Gatifloxacin, Levofloxacin, Lomefloxacin, Moxifloxacin, Norfloxacin, Ofloxacin, and Trovafloxacin.

Representative sulfonamides include Mafenide, Prontosil, Sulfacetamide, Sulfamethizole, Sulfanilimide, Sulfasalazine, Sulfisoxazole, Trimethoprim, and Trimethoprim-Sulfamethoxazole (Co-trimoxazole) (TMP-SMX).

Representative tetracyclines and tetracycline-like compounds include Glycylcycline class antibiotics such as Tigecycline; Demeclocycline, Doxycycline, Minocycline, Oxytetracycline, and Tetracycline.

Other antibacterial agents include, for example, Arsphenamine, Chloramphenicol, Clindamycin, Lincomycin, Ethambutol, Fosfomycin, Fusidic acid, Furazolidone, Isoniazid, Linezolid, Metronidazole, Mupirocin, Nitrofurantoin, Platensimycin, Pyrazinamide, Quinupristin/Dalfopristin, Rifampin or Rifampicin, and Tinidazole.

III. Methods of Using the Compounds and/or Pharmaceutical Compositions

The compounds can be used to treat or prevent microbial infections caused by Staphylococcus aureus. The compounds can also be used as adjunct therapy in combination with existing therapies in the management of the aforementioned types of infections. In such situations, it is preferably to administer the active ingredients to a patient in a manner that optimizes effects upon the Staphylococcus aureus bacteria, including drug resistant versions, while minimizing effects upon normal cell types. While this is primarily accomplished by virtue of the behavior of the compounds themselves, this can also be accomplished by targeted drug delivery and/or by adjusting the dosage such that a desired effect is obtained without meeting the threshold dosage required to achieve significant side effects.

S. aureus propagation can be inhibited by inhibiting ribosomal binding of a specific tRNA useful for incorporation of arginine into a growing peptide or protein in S. aureus, by an amount sufficient to inhibit S. aureus propagation. Inhibition of ribosomal binding may be carried out by contacting an active compound to the ribosome in an amount effective to inhibit binding sufficiently to inhibit S. aureus propagation. The S. aureus may be in vitro, in a culture media, or on a surface to be disinfected, or may be in vivo in a host (e.g., a human or animal host in need of an antimicrobial treatment). Formulations of active compounds can be prepared and administered in accordance with known techniques, as discussed below.

A method of inhibiting S. aureus propagation in a host comprises inhibiting the binding of the specific host tRNA to the S. aureus RNA at one of the binding sites by an amount sufficient to inhibit propagation of the S. aureus in the host.

Formulations of active compounds can be prepared and administered in accordance with known techniques, as discussed below. In a preferred embodiment, the specific host tRNA is tRNA^{Arg}. Preferably the S. aureus primes translation specifically with the specific host tRNA, such as tRNA^{arg}_{mnm5UCU}. The host may be a cell in vitro, or a human or animal subject in need of such treatment.

Subjects to be treated by the methods of the present invention are typically human subjects although the methods may be carried out with animal subjects (dogs, cats, horses, cattle, etc.) for veterinary purposes. The present invention provides pharmaceutical formulations comprising the active compounds, including pharmaceutically acceptable salts thereof, in pharmaceutically acceptable carriers for aerosol, oral, and parenteral administration as discussed in greater detail below. The therapeutically effective dosage of any specific compound, the use of which is in the scope of present invention, will vary somewhat from compound to compound, patient to patient, and will depend upon the condition of the patient and the route of delivery.

In accordance with the present method, an active compound or a pharmaceutically acceptable salt thereof, may be administered orally or through inhalation as a solid, or may be administered intramuscularly or intravenously as a solution, suspension, or emulsion. Alternatively, the compound or salt may also be administered by inhalation, intravenously or intramuscularly as a liposomal suspension. When administered through inhalation the active compound or salt should be in the form of a plurality of solid particles or droplets having a particle size from about 0.5 to about 5 microns, preferably from about 1 to about 2 microns.

The present invention will be better understood with reference to the following non-limiting example.

### EXAMPLE 1

### Working Assay Protocol for TRANA SA 101 HTS Assay

The following is a general example of the screening assay described herein.

Assay Target: Ribosome of SA programmed with Arg message.
Assay tool: ASL of SA tRNA Arg with FL label
Sample detection method:—Currently fluorescence quenching but will convert to time resolved fluorescence polarization.
Sample format 96 and 384 well plates

Sample volume: 100 μl and 5 μl

Target concentration: 5 pM

Tool concentration: 10 pM

DMSO concentration 10%

### Materials

Isolated Ribosome (2 pM/μl)

Message 2 ug/μl

ASL 3-6 ug/μl

CMN buffer—80 mM potassium cacodylate, pH 7.2, 20 mM MgCl₂, 100 mM NH₄Cl, and 3 mM β-mercaptoethanol

Method—96 well format

1. Prepare programmed ribosome as follows:
a. Mix 5 pM per well,
b. 2 μg message
c. 0.3-10 pM ASL
d. 10 μl test compound
e. 100 μl volume

2. 20 min complex formation @ 37° C.

3. Read fluorescence on plate reader

Literature Cited

The following references were cited herein, and the contents of these references, and all other references cited herein, are hereby incorporated by reference in their entirety.

Agris P F. Decoding the genome: a modified view. Nucleic Acids Res. 2004 Jan. 9; 32(1):223-38. Print 2004. Review.

Agris P F, Malkiewicz A, Kraszewski A, Everett K, Nawrot B, Sochacka E, Jankowska J, Guenther R. Biochimie. (1995) Site-selected introduction of modified purine and pyrimidine ribonucleotides into RNA by automated phosphoramidite chemistry. 77(1-2):125-34.

Agris P F, Guenther R, Ingram P C, Basti M M, Stuart J W, Sochacka E, Malkiewicz A. (1997) Unconventional structure of tRNA(Lys)SUU anticodon explains tRNA's role in bacterial and mammalian ribosomal frameshifting and primer selection by HIV-1. RNA. 1997 April; 3(4):420-8.

Agris P F, Guenther R, Sochacka E, Newman W, Czerwinska G, Liu G, Ye W, Malkiewicz A. (1999) Thermodynamic contribution of nucleotide modifications to yeast tRNA(Phe) anticodon stem loop analogs. Acta Biochim Pol. 1999; 46(1):163-72.

Ashraf S S, Sochacka E, Cain R, Guenther R, Malkiewicz A, Agris P F. RNA (1999) Single atom modification (O->S) of tRNA confers ribosome binding. (2):188-94.

Ashraf S S, Ansari G, Guenther R, Sochacka E, Malkiewicz A, Agris P F. (1999) The uridine in “U-turn”: contributions to tRNA-ribosomal binding. RNA. 1999 April; 5(4):503-11.

CDC 2000-2001. “Drug Resistance/Antimicrobial Resistance”, Centers for Disease Control and Prevention, CDC Fact Book 2000/2001, p. 75.

DRAGON. http://www.disat.unimib.it/chm/Dragon.htm. 2005. Ref Type: Electronic Citation

Francois B, Russell R J, Murray J B, Aboul-ela F, Masquida B, Vicens Q, Westhof E. Nucleic Acids Res. (2005) Crystal structures of complexes between aminoglycosides and decoding A site oligonucleotides: role of the number of rings and positive charges in the specific binding leading to miscoding. 33 (17):5677-90.

Grosjean, H. and Benne, R. Modification and editing of RNA. Washington, DC: ASM Press, c1998.

Hermann T. Curr Opin Struct Biol. (2005) Drugs targeting the ribosome. 15(3):355-66.

Kumar R K, Davis D R. Synthesis and studies on the effect of 2-thiouridine and 4-thiouridine on sugar conformation and RNA duplex stability. Nucleic Acids Res. 1997 Mar. 15; 25(6):1272-80.

Limbach, P. A., P. F. Crain and J. A. McCloskey. Summary: the modified nucleotides of RNA. Nucleic Acids Res. 22, 2183-2196 (1994).

Malkiewicz, A. and E. Sochacka 1983 The protected derivatives of 5-methylaminomethyl-2-thiouridine and 5-carbomethoxymethyl-2-thiouridine as components for the oligonucleotide synthesis. Tetrahedron Letters 24, 5387-5390.

Murphy F V 4th, Ramakrishnan V, Malkiewicz A, Agris P F. Nat Struct Mol Biol. (2004) The role of modifications in codon discrimination by tRNA(Lys)UUU. (12):1186-91.

Nobles K N, Yarian C S, Liu G, Guenther R H, Agris P F. (2002) Highly conserved modified nucleosides influence Mg2+-dependent tRNA folding. Nucleic Acids Res. 30:4751-60.

Ogilvie K K, Usman N, Nicoghosian K, Cedergren R J. 1988 Total chemical synthesis of a 77-nucleotide-long RNA sequence having methionine-acceptance activity. Proc Natl Acad Sci USA. 85:5764-8

Phelps S S, Malkiewicz A, Agris P F, Joseph S. J Mol Biol. (2004) Modified nucleotides in tRNA(Lys) and tRNA(Val) are important for translocation 338(3):439-44.

PubChem. http://pubchem.ncbi.nlm.nih.gov/2008. Ref Type: Electronic Citation

Ryszard W. Adamiak and Jacek Stawinski, 1977. A highly effective route to N,N′-disubstituted ureas under mild conditions. an application to the synthesis of tRNA anticodon loop fragments containing ureidonucleosides. Tetrahedron Letters, 18: 1935-1936.

Schilling-Bartetzko S, Franceschi F, Sternbach H, Nierhaus K H. Apparent association constants of tRNAs for the ribosomal A, P, and E Sites. J Biol Chem. 1992 Mar. 5; 267(7):4693-702.

Soll, D. and RajBhandary, U. L. tRNA: structure, biosynthesis, and function. ASM Press, c1995.

Stuart J W, Gdaniec Z, Guenther R, Marszalek M, Sochacka E, Malkiewicz A, Agris P F. (2000). Functional anticodon architecture of human tRNALys3 includes disruption of intraloop hydrogen bonding by the naturally occurring amino acid modification, t6A.Biochemistry. 39:13396-404.

Sundaram M, Crain P F, Davis D R. 2000. Synthesis and characterization of the native anticodon domain of E. coli tRNA(Lys): simultaneous incorporation of modified nucleosides mnm(5)s(2)U, t(6)A, and pseudouridine using phosphoramidite chemistry. J Org Chem. 65:5609-14.

Tropsha, A, Golbraikh, A. 2007. Predictive QSAR Modeling Workflow, Model Applicability Domains, and Virtual Screening. Curr. Pharm. Des. 13: 3494-504

von Ahsen U, Green R, Schroeder R, Noller H F. Identification of 2′-hydroxyl groups required for interaction of a tRNA anticodon stem-loop region with the ribosome.RNA. 1997 January; 3(1):49-56.

Wells B D, Cantor C R. 1980. Ribosome binding by tRNAs with fluorescent labeled 3′ termini. Nucleic Acids Res. 1980 Jul. 25; 8(14):3229-46.

Yarian C, Townsend H, Czestkowski W, Sochacka E, Malkiewicz A J, Guenther R, Miskiewicz A, Agris P F. J Biol Chem. (2002) Accurate translation of the genetic code depends on tRNA modified nucleotides. 277(19):16391-5.

Zhang J. H., Chung T. D. Y., Oldenburg, K. R J. Biomol. Screen (1999) A simple statistical parameter for use in the evaluation and validation of high throughput screening assays. 4, 67-73.

Zhang R, Ou H Y, Zhang C T. Nucleic Acids Res. 2004 Jan. 1; 32 (Database issue):D271-2. DEG: a database of essential genes.

Although the foregoing invention has been described in some detail by way of illustration and example for purposes of clarity of understanding, it will be obvious that certain changes and modifications may be practiced within the scope of the appended claims.

1. An isolated nucleic acid molecule comprising the nucleic acid sequence
AUGGCs2CUmnm5UCUt6AAGCCAU-label

2. The isolated nucleic acid molecule of claim 1, comprising the nucleic acid sequence AUGGCs2CUmnm5UCUt6AAGCCAU-Fluorescein.

3. The isolated nucleic acid isolated nucleic acid molecule of claim 1, wherein the label is detectable, either directly or indirectly, by spectroscopic, photochemical, biochemical, immunochemical, or chemical means.

4. The method of claim 1, wherein the label is selected from the group consisting of radioactive isotopes (for example, ³²P, ³⁵S, and ³H), dyes, fluorescent dyes (for example, Cy5 and Cy3), fluorophores (for example, fluorescein), electron-dense reagents, enzymes and their substrates (for example, as commonly used in enzyme-linked immunoassays, such as, alkaline phosphatase and horse radish peroxidase), biotin-streptavidin, digoxigenin, or hapten; and proteins for which antisera or monoclonal antibodies are available.

5. The isolated nucleic acid molecule of claim 1, wherein the label is an affinity tag.

6. A method of capturing the isolated tRNA fragment of claim 5, comprising contacting the isolated tRNA fragment of claim 1, wherein the label is an affinity tag, with a complimentary ligand coupled to a solid support that allows for the capture of the affinity tag-labeled tRNA fragment.

7. The method of claim 6, wherein the affinity tags and complimentary partners are selected from the group consisting of biotin-streptavidin, complimentary nucleic acid fragments (for example, oligo dT-oligo dA, oligo T-oligo A, oligo dG-oligo dC, oligo G-oligo C), aptamers, or haptens and proteins for which antisera or monoclonal antibodies are available.

8. A method of identifying a specific inhibitor of RNA translation in Staphylococcus aureus, comprising:
contacting, in the presence of a test compound, a nucleic acid molecule consisting essentially of a nucleic acid sequence encoding an anticodon stem loop having the nucleic acid sequence of claim 1 to a ribosome capable of binding to the nucleic acid molecule;
incubating under conditions that allow binding of the nucleic acid molecule and the ribosome in the absence of the test compound, and
detecting the inhibition of binding of the nucleic acid molecule and the ribosome by the test compound.

9. The method of claim 8, wherein:
a) the nucleic acid molecule forms a complex with arginine and with the ribosome, and
b) the binding between the nucleic acid molecule and the ribosome is specifically related to arginine incorporation into a peptide or protein in S. aureus, but not arginine incorporation in the majority of other bacteria.

10. The method of claim 8, wherein the nucleic acid molecule is the nucleic acid molecule of claim 1.

11. The method of claim 8, wherein the nucleic acid molecule is the nucleic acid molecule of claim 2.

12. A method of screening for compounds useful for specifically inhibiting S. aureus propagation, comprising: contacting, in the presence of a test compound, a specific S. aureus tRNA that is unique to S. aureus and not present in the majority of other bacteria, to a ribosome that is ordinarily capable of binding said tRNA, wherein said contacting step is carried out in vitro; and then determining whether said compound inhibits the binding of said tRNA to said ribosome; the inhibition of binding indicating said test compound is useful for inhibiting S. aureus propagation.

13. A method according to claim 12, wherein said determining step comprises determining whether said compound inhibits the binding of said tRNA to said ribosome at position 27-43 of said tRNA.

14. A method according to claim 12, wherein said tRNA is tRNA^{arg}_{mnm5UCU}.


SEQUENCE LISTING (1 sequence)

SEQ ID NO: 1 - RNA, 17 nt, S. aureus
  modified_base (6)..(6): s2c
auggccuucuaagccau